(19) United States
(12) Patent Application Publication
(10) Pub. No.: US 2021/0169703 A1
(43) Pub. Date: Jun. 10, 2021

(54) Barrier Band

(71) Applicant: Lorri Lynn Izzo, Sparta, NJ (US)

(72) Inventor: Lorri Lynn Izzo, Sparta, NJ (US)

(21) Appl. No.: 16/537,922

(22) Filed: Aug. 12, 2019

(51) Int. Cl. A61F 13/10; A61M 39/02; A61F 13/06

(52) U.S. Cl. CPC A61F 13/10; A61M 39/02; A61F 2013/00093; A61M 2039/0205; A61F 13/06

(57) ABSTRACT

The Barrier Band for an area on a human body, involving a vascular device access area. The present invention relates to an apparatus that provides a barrier between a person's skin, and any vascular access device, and its components, to prevent compromising skin and to provide comfort to a person. The actual barrier is provided in several variations. The Barrier Band is offered in both a washable and disposable version, and its size, style and stitching, if any, are offered in a variety ways, which is determined by a person's weight and what body part is being covered. It may or may not have an adjustable apparatus or any other suitable component to tighten or loosen the band.

### CROSS-REFERENCE TO RELATED APPLICATIONS

The application is a continuation in part and claims the benefits of U.S. Non-Provisional application Ser. No. 16/537,922, filed Aug. 12, 2019.

### BACKGROUND OF THE INVENTION

### 1. Field of Invention

This invention relates generally to a vascular access device site, and more specifically to a cover with which can assist in preventing such a site from compromising someone's skin. When a patient has any type of vascular access device is covered with a medical grade gauze, netting, spandex, or other suitable material. This is to keep it secure. A problem that many patients have when they are required to wear this cover is that their skin becomes irritated as a result of the cover causing the vascular access device and its components to press against the skin. Consequently, whether it is short or long term use, the skin should be protected during its use, and no current invention provides a barrier between the skin and vascular access device and its components, like the present invention does. At times patients that need this device already have comprised skin, which means it will be further compromised. This complete coverage fully eliminates all contact. The band's specific measurement and configuration is determined by a persons weight, and which body part it is utilized. A common complaint amount people is that with extended use, a cover of this type does not work effectively. For that reason, some variations of the Barrier Band have an adjustable apparatus including: zips, hooks, toggles, elastic, snaps, zippers, velcro, or any other suitable component present. This adjustable apparatus is implemented in an effort to tighten or loosen the band.

### SUMMARY OF INVENTION

It is an object of the present invention to provide an easily-installable means for protecting a vascular access device area on a human body. It is another object of the present invention to achieve this using multiple sizes so that it may fit the various sizes of human extremities. These and other objects and advantages of the invention will become readily apparent as the following description is read in conjunction with the accompanying drawings. The present invention relates to a band apparatus that provides a barrier with a ductway between the skin on any part of a person's body and a vascular access device and its components. It is utilized to prevent skin from being compromised, eliminate discomfort, and keep the vascular access device and its components secure. The Barrier Band is constructed in one of two ways. One way is a breathable, disposable mesh, gauze, netting or any other suitable disposable material. The other is a breathable, washable, spandex or any other suitable washable material. With usage and a person's weight fluctuation a person could need to tighten or loosen the Barrier Band, and that is why some versions of the Barrier Band offer built in adjustable apparatuses.

There are several ways that the method of the barrier is delivered. This variant is determined by a person's weight, and where the Barrier Band will be located on their body. The band in one embodiment has a ductway opening on one end and at least another ductway opening at the other end integrally formed in the sleeve, which form a barrier between the person's skin and any vascular access device and its components. This band's midsection contains stitching defining the ductway. The band is pliable and cylindrical in shape with two open ends. Another embodiment has the same features, and has only one stitch to secure the canal. The length of stitching on a Barrier Band can vary in size. Stitching can be replaced by glue or any suitable material that could adhere the ductway to the band. An area of the band or stitching might be a different color to for instructional purposes. Another embodiment has containing no stitching, and is essentially a band within a band, acting as a double layer, still providing the barrier between the skin and vascular access device and its components. Another embodiment is flat opened version of the present invention, and involves a wrapping technique. The Barrier Band may or may not have an adjustable apparatus including: zips, hooks, toggles, elastic, snaps, zippers, velcro, or any other suitable component present. This adjustable apparatus is implemented in an effort to tighten or loosen the band. The band's specific measurement is determined by a persons weight, and which body part it is utilized. The presence of stitching or other suitable material and its sizing, style and location on a Barrier Band, will vary among the variations.

### OBJECT OF THE INVENTION

It is an object of the present invention to protect the skin of someone who has a vascular access device and/or its components placed on any body part with a band like apparatus. It is another object of the invention to protect such skin without dragging, snagging, pulling or otherwise interfering with the vascular access device and its components. A feature of this invention is that the band is made from a single piece of washable or disposable material that is smooth on the inner and outer surface, includes an opening in the fore and aft ends, and contains a ductway located at each end, which provides a barrier between the skin and the components of the vascular access device. In another embodiment stitching is present adjacent the aft and fore ends defining the ductway. In another embodiment the embodiment is doubled layered and contains no stitching. The size or presence of stitching or any others means of adherence, and its positioning will vary among different embodiments. In another embodiment an adjustable apparatus (i.e. zips, hooks, toggles, elastic snaps, zippers etc) or any other suitable adjustable apparatus component is present. This adjustable apparatus is implemented in an effort to tighten or loosen the band. Any band's specific measurement is determined by a person's weight, and which body part it is covered.

### BRIEF DESCRIPTION OF THE DRAWING

The present invention, in accordance with one or more various embodiments, is described in detail with reference to the following figures. The drawings are provided for purposes of illustration only and merely depict typical or example embodiments of the invention. In an effort to have the reader understand the invention and not to limit the breadth, scope, or applicability of the invention in any way, these drawings are provided. These drawings are not necessarily made to scale. Descriptive drawing terms illustrating various embodiment-viewing angles, such as “top,” or “front” views are merely descriptive and do not imply or require that the invention be implemented or used in a particular spatial orientation unless explicitly stated otherwise.

FIG. 1 is a front and top view of the present invention with a ductway containing a double stitched configuration.

FIG. 2 is a top view taken over line 1 of FIG. 1.

FIG. 3 is a front and top view of the present invention with a ductway containing a double stitched configuration, featuring an adjustable apparatus;

FIG. 4 is a front view of the adjustable apparatus.

FIG. 5 is a top view taken over line 1 of FIG. 3.

FIG. 6 is a front and top view of the present invention with a ductway containing a double stitched configuration featuring an adjustable hook apparatus.

FIG. 7 is a front view of the adjustable full hook apparatus.

FIG. 8 is a front view of the adjustable hook apparatus, as it would appear attached to the present invention.

FIG. 9 is a top view taken over line 1 of FIG. 6.

FIG. 10 is a front and top view of the present invention with two ductways containing a single stitch configuration.

FIG. 11 is a top view taken over line 1 of FIG. 10.

FIG. 12 is a front and top view of the present invention with two ductways containing a single shortened stitch configuration.

FIG. 13 is a top view taken over line 1 of FIG. 12.

FIG. 14 is a front and top view of the present invention with two ductways, containing a no stitch configuration.

FIG. 15 is a top view taken over line 1 of FIG. 14.

FIG. 16 is a cut-through view of the present invention with a curved ductway entry point.

FIG. 17 is a cut-through view of the present invention with a flat ductway entry point.

FIG. 18 is a is a front and top view of the present invention in an opened version containing a foldable ductway and foldable band configuration.

FIG. 19 is a side view taken over line 1 of FIG. 18.

FIG. 20 is a cut through view of line 1 of FIG. 18.

FIG. 21 is another perspective view of the invention positioned over a hand.

FIG. 22 is another perspective view of the invention positioned over a foot.

FIG. 23 is another perspective view of the invention positioned over a ankle.

FIG. 24 is another perspective view of the invention positioned over a neck.

FIG. 25 is another perspective view of the invention positioned over a head.

FIG. 26 is another perspective view of the invention positioned over a arm.

FIG. 27 is another perspective view of the invention positioned over a chest.

FIG. 28 is another perspective view of the invention positioned over a leg.

### DETAILED DESCRIPTION OF THE PREFERRED EMBODIMENTS

Various embodiments are now described with reference to the drawings, wherein like reference numerals are used to refer to like elements throughout. In the following description, for purposes of explanation, specific details are set forth in order to provide a thorough understanding of one or more embodiments. It may be evident, however, that such embodiment(s) may be practiced without these specific details and be practiced with modification within the spirit and cope of the appended claims. The present invention is directed toward a band that covers a person's vascular access device area on their body, while simultaneously protecting their skin with a barrier. When a patient has any type of vascular access device it is covered with a medical grade gauze, netting, spandex, or other suitable material. This is to keep it protected and secure. A problem that many patients have when they are required to wear this cover is that their skin becomes irritated as a result of the cover causing the vascular access device and its components to press against the skin. Consequently, whether it is short or long term use, the skin should be protected during its use, and no current invention provides a barrier between the skin and vascular access device and its components, like the present invention does. At times patients that need this device already have comprised skin, which means it will be further comprised. This complete coverage fully eliminates all discomfort. The band's specific measurement and configuration is determined by a person's weight, and which body part it is utilized. A common complaint amount people is that with extended use, a cover of this type does not work effectively. For that reason, some variations of the Barrier Band have an adjustable apparatus including: zips, hooks, toggles, elastic, snaps, zippers, velcro, or any other suitable component present. This adjustable apparatus is implemented in an effort to tighten or loosen the band.

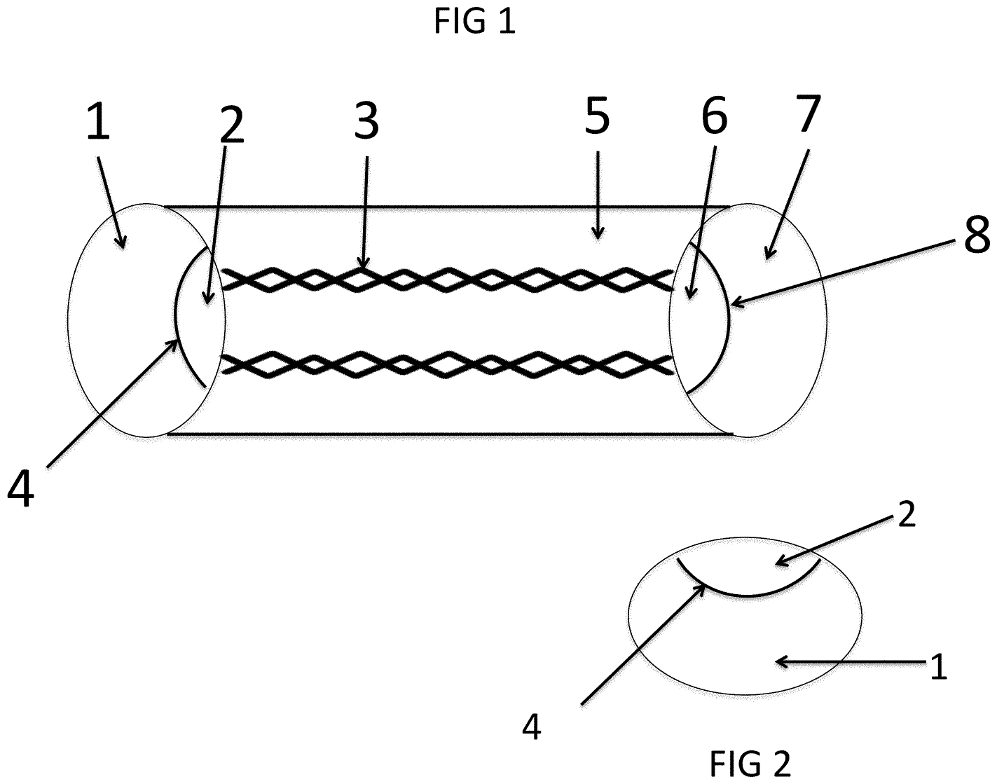

FIG. 1 Illustrates a band (5) for a vascular access device site. Such a site can be present on several different body parts. The band (5) can be placed on a body part through the open ends (1) and (8), and the band's specific measurement is determined by a patient's size, and which body part it is being covered. This is a front and top view of the present invention featuring a ductway opening (2) and (6) that creates a barrier (4) and (8) between a person's vascular access device and their skin. This embodiment contains a double stitch (3) configuration.

FIG. 2 Is a top view taken over line 1 of FIG. 1. It illustrates an opening (1) for a body part, the ductway (2) where the vascular access device components are placed, and the actual barrier (4) provided by the Barrier Band, in between the body part and the vascular access device, and its components.

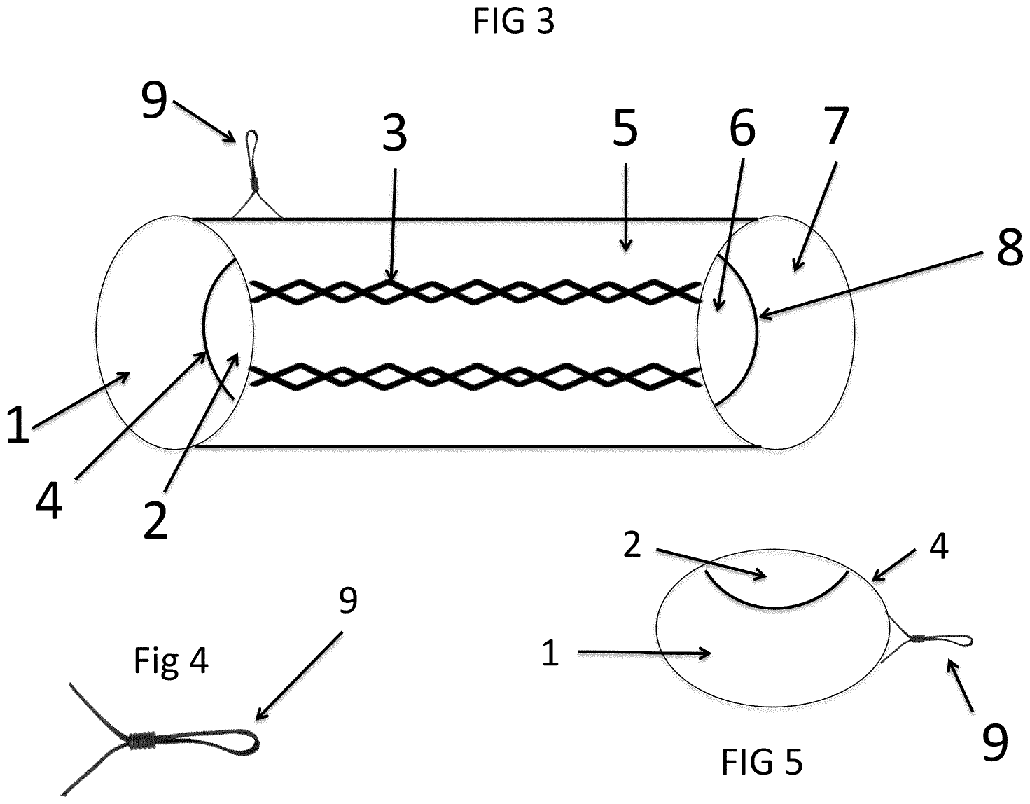

FIG. 3 Illustrates a band (5) for a vascular access device site. Such a site can be present on several different body parts. The band (5) can be placed on a body part through the open ends (1) and (8), and the band's specific measurement is determined by a patient's size, and which body part it is being covered. This is a front and top view of the present invention featuring a ductway opening (2) and (6) that creates a barrier (4) and (8) between a person's vascular access device and their skin. This embodiment contains a double stitch (3) configuration, and features an adjustable apparatus (9), in the form of a elastic pull string, which is inserted to enable someone to tighten or loosen their band.

FIG. 4 is a front view of the adjustable apparatus (9), which is inserted to enable someone to tighten or loosen their band.

FIG. 5 is a top view taken over line 1 of FIG. 3. It illustrates an opening (1) for a body part, the ductway (2) where the vascular access device components are placed and the actual barrier (4) in between the body part and the vascular access device, and its components. It also features an adjustable apparatus (9), in the form of a elastic, which is inserted to enable someone to tighten or loosen their band.

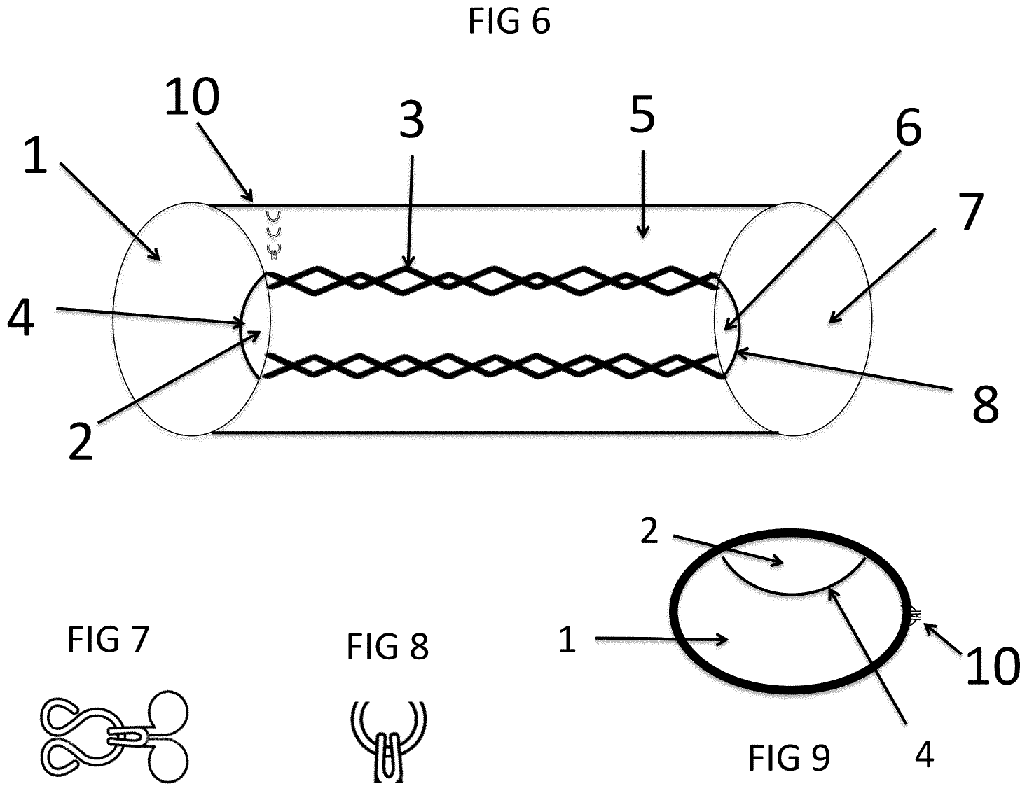

FIG. 6 Illustrates a band (5) for a vascular access device site. Such a site can be present on several different body parts. The band (5) can be placed on a body part through the open ends (1) and (8), and the band's specific measurement is determined by a patient's size, and which body part it is being covered. This is a front and top view of the present invention featuring a ductway opening (2) and (6) that creates a barrier (4) and (8) between a person's vascular access device and their skin. This embodiment contains a double stitch (3) configuration, and features an adjustable apparatus (10) in the form of a hook, which is inserted to enable someone to tighten or loosen their band.

FIG. 7 is a front view of the adjustable full hook apparatus. This is the hook in its full form before it is adhered to the band (5).

FIG. 8 is a front view of the adjustable hook apparatus, as it would appear when it is attached to the present invention.

FIG. 9 is a top view taken over line 1 of FIG. 6. It illustrates an opening (1) for a body part, the ductway (2) where the vascular access device components are placed and the actual barrier (4) in between the body part and the vascular access device, and its components. It also features an adjustable apparatus (9), in the form of a hook, which is inserted to enable someone to tighten or loosen their band.

FIG. 10 Illustrates a band (5) for a vascular access device site. Such a site can be present on several different body parts. The band (5) can be placed on a body part through the open ends (1) and (8), and the band's specific measurement is determined by a patient's size, and which body part it is being covered. This is a front and top view of the present invention featuring a ductway opening (2) and (6) that creates a barrier (4) and (8) between a person's vascular access device and their skin. This embodiment contains a single stitch (3) configuration.

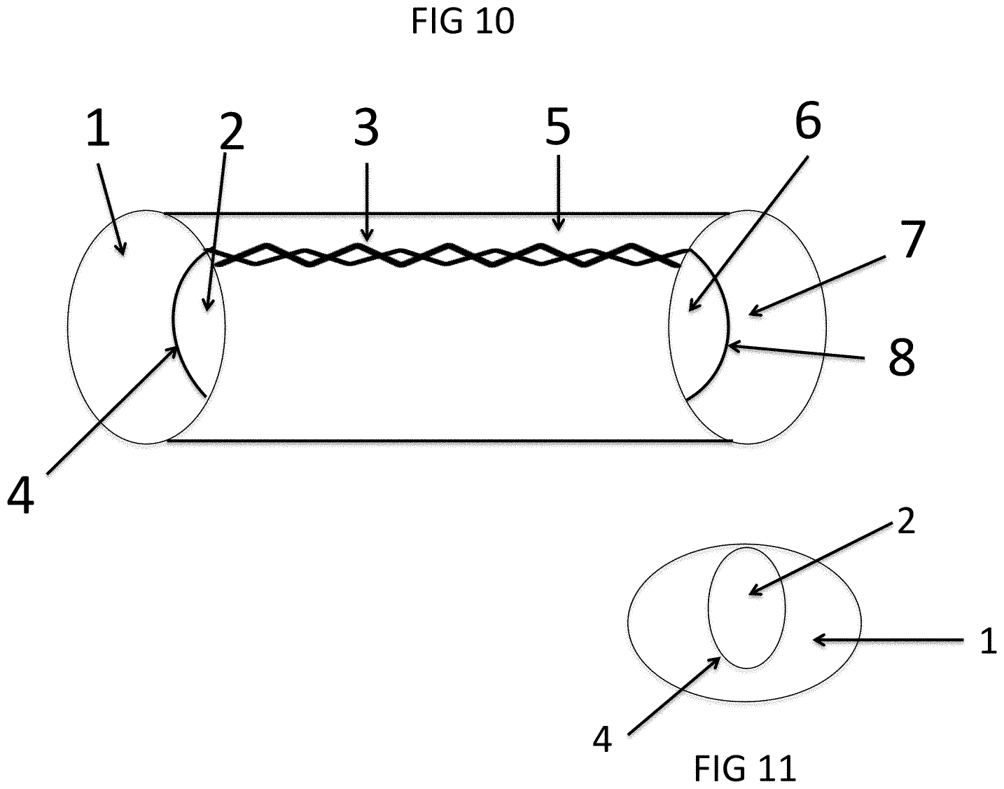

FIG. 11 Is a top view taken over line 1 of FIG. 10. It illustrates an opening (1) for a body part, the ductway (2) where the vascular access device components are placed, and the actual barrier (4) provided by the Barrier Band, in between the body part and the vascular access device, and its components.

FIG. 12 Illustrates a band (5) for a vascular access device site. Such a site can be present on several different body parts. The band (5) can be placed on a body part through the open ends (1) and (8), and the band's specific measurement is determined by a patient's size, and which body part it is being covered. This is a front and top view of the present invention featuring a ductway opening (2) and (6) that creates a barrier (4) and (8) between a person's vascular access device and their skin. This embodiment contains a shortened single stitch (3) configuration.

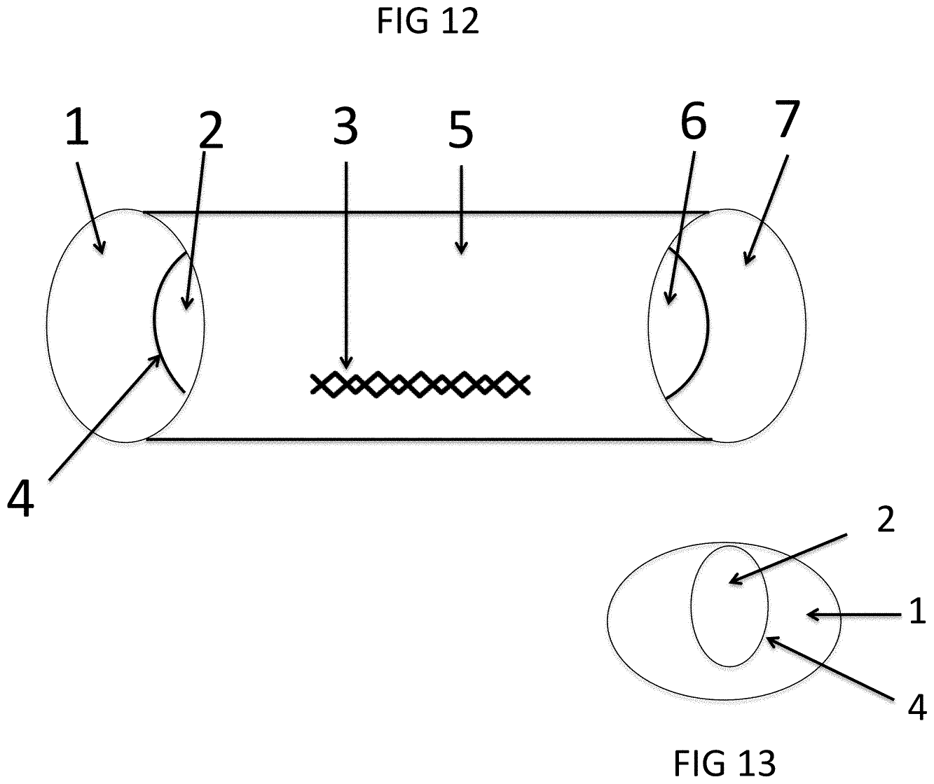

FIG. 13 Is a top view taken over line 1 of FIG. 12. It illustrates an opening (1) for a body part, the ductway (2) where the vascular access device components are placed, and the actual barrier (4) provided by the Barrier Band, in between the body part and the vascular access device, and its components.

FIG. 14 Illustrates a band (5) for a vascular access device site. Such a site can be present on several different body parts. The band (5) can be placed on a body part through the open ends (1) and (7), and the band's specific measurement is determined by a patient's size, and which body part it is being covered. This is a front and top view of the present invention featuring a ductway opening (2) and (6) that creates a barrier (4) and (8) between a person's vascular access device and their skin. This embodiment contains no stitch.

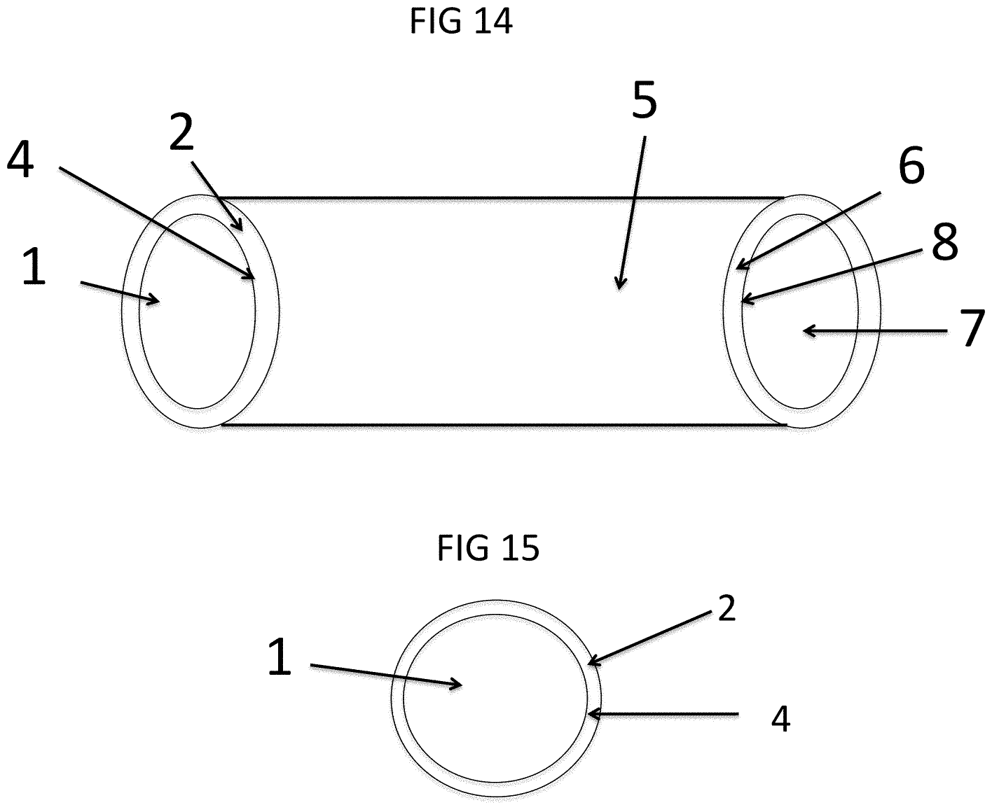

FIG. 15 Is a top view taken over line 1 of FIG. 14. It illustrates an opening (1) for a body part, the ductway (2) where the vascular access device components are placed, and the actual barrier (4) provided by the Barrier Band, in between the body part and the vascular access device, and its components.

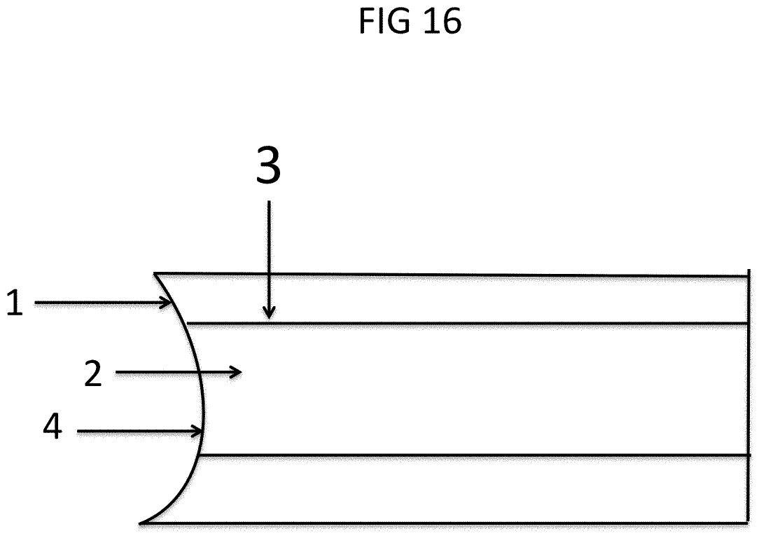

FIG. 16 is a cut-through view of the present invention showing the access point for a body part (1) with a curved ductway entry point (4), stitching (3), and a open view of the barrier (2).

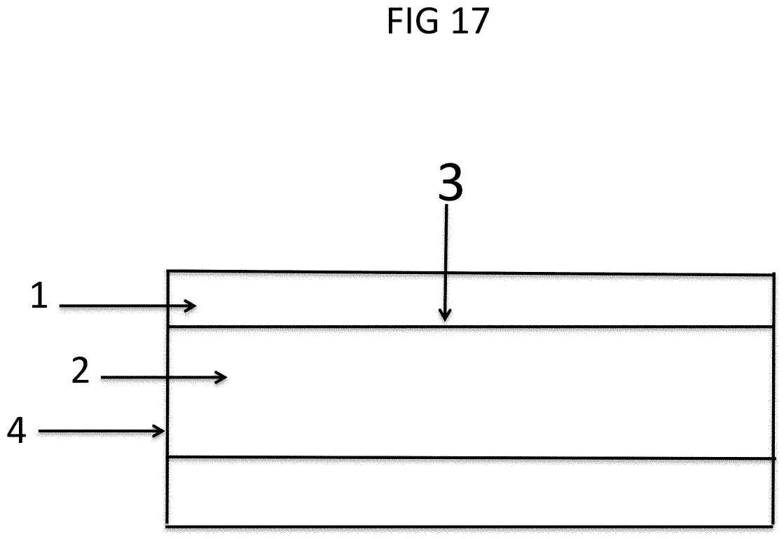

FIG. 17 is a cut-through view of the present invention showing the access point for a body part (1) with a flat ductway entry point (4), stitching (3), and a open view of the barrier (2).

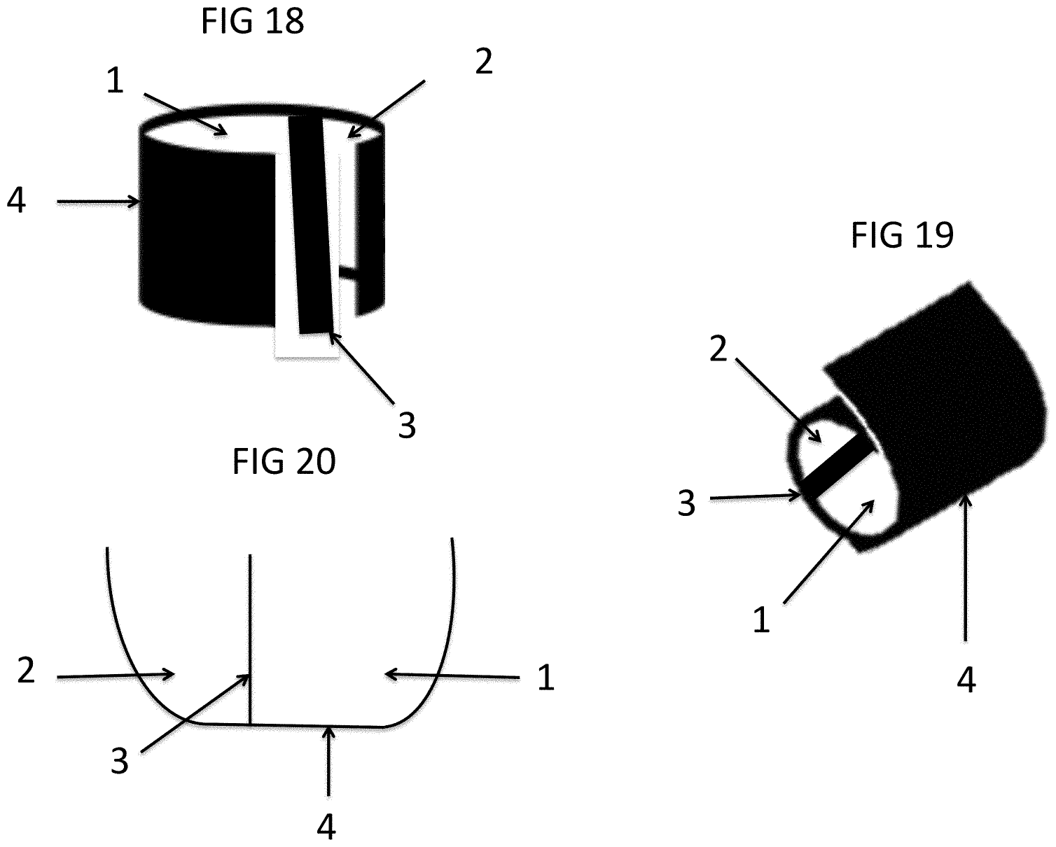

FIG. 18 is a is a front and top view of the present invention, where the band (4) is offered in an opened version configuration, containing a foldable ductway (3) and the band (4) can be folded over a body part. In this embodiment, the body part is placed on one side (1) of the ductway, and the components of the vascular access device are placed on the other side (2) of the ductway.

FIG. 19 is a side view taken over line 1 of FIG. 18 of the present invention, where the band (4) is offered in an opened version configuration, containing a foldable ductway (3) and the band (4) can be folded over a body part. In this embodiment, the body part is placed on one side (1) of the ductway, and the components of the vascular access device are placed on the other side (2) of the ductway.

FIG. 20 is a cut through view of line 1 of FIG. 18 of the present invention, where the band (4) is offered in an opened version configuration, containing a foldable ductway (3) and the band (4) can be folded over a body part. In this embodiment, the body part is placed on one side (1) of the ductway, and the components of the vascular access device are placed on the other side (2) of the ductway.

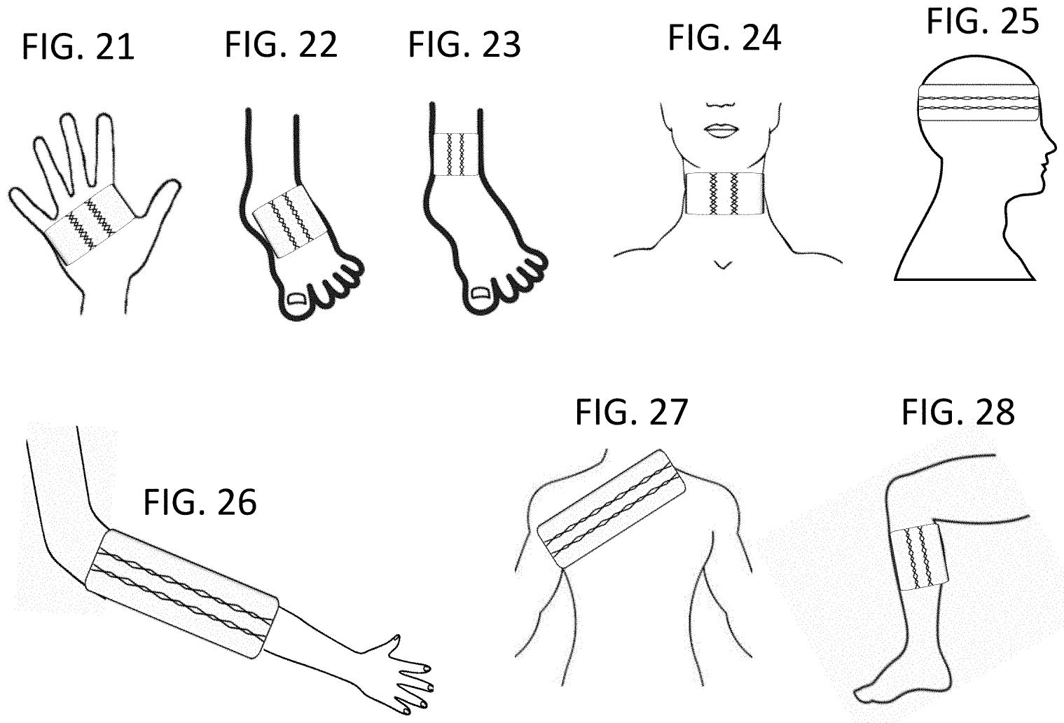

FIG. 21 is another perspective view of the invention in use and positioned over a hand.

FIG. 22 is another perspective view of the invention in use and positioned over a foot.

FIG. 23 is another perspective view of the invention in use and positioned over a ankle.

FIG. 24 is another perspective view of the invention in use and positioned over a neck.

FIG. 25 is another perspective view of the invention in use and positioned over a head.

FIG. 26 is another perspective view of the invention in use and positioned over a arm.

FIG. 27 is another perspective view of the invention in use and positioned over a chest.

FIG. 28 is another perspective view of the invention in use and positioned over a leg.

The body parts listed and illustrated in FIGS. 21 through 28, are for the intent to provide an understanding of the invention's purpose, and not to limit its use on those body parts only. As illustrated and described above, through the several embodiments offered there are many effective ways to implement the barrier's method of use regardless of a person's weight or what body part needs to be covered.

I claim:

1. A band for a vascular device access area on a human body compromising:
a main body, said main body being comprised of a band having two open ends and a ductway, made from a single piece and having a constant thickness made from a cloth material.
a first portion of said main body to form around a human body part at one said end of said band;
a second portion of said main body to form around a human body part at a second open end of said band;
the circumference of said first and second portions having equal sizes;
a third portion of said main body wherein a first ductway opening is integrally formed at said first open end and a second ductway opening is integrally formed at said second end.

2. The cloth material as claimed in claim 1, wherein said cloth material is a disposable material.

3. The cloth material as claimed in claim 1, wherein said cloth material is a washable fabric.

4. The ductway as claimed in claim in 1, wherein said ductway is attached with stitching.

5. The stitching in claim 4, wherein said stitching is on both sides of the ductway.

6. The stitching in claim 4, wherein said stitching is on one side of the ductway.

7. The stitching in claim 6, wherein said stitching is shortened by half the size.

8. The ductway in claim 1 wherein said ductway has no stitching.

9. The ductway in claim 1 wherein said ductway is adhered to the band using an adhesive.

10. The stitching as claimed in claim 4, wherein a portion of said stitching is defined with a color.

11. The circumference in claim 1, wherein said circumference does not have equal portions at each end.

12. The band in claim 1, wherein said band has an adjustable apparatus

13. The adjustable apparatus in claim 11, wherein said adjustable apparatus is a hook.

14. The adjustable apparatus in claim 11, wherein said adjustable apparatus is elastic.

15. The material in claim 1 where said material is offered in a variety of colors and/or patterns.

16. The band in claim 1, wherein said band has a colored area.

17. The ductway opening as claimed in claim in 1, wherein said ductway opening has a curvature.

18. The ductway opening as claimed in claim in 1, wherein said ductway opening has no curvature.

19. The band in claim 1, wherein said band is in an open configuration, containing an attachable component.

20. A method of protecting the skin on any area of a human body, surrounding a vascular device access area, using a washable fabric or disposable material, formed in an open or continual band, opened at both ends, containing a ductway, creating a barrier between the skin and a vascular access device and its components comprising the steps of:
i. placing said band on a body part;
ii. adjusting said band up to the vascular device access area;
iii. placing the vascular device access components into the band's ductway;
iv. moving the band's end further up a portion of said area positioning it to cover said vascular device access area entirely.
